(19) United States
(12) Patent Application Publication
(10) Pub. No.: US 2010/0272805 A1
(43) Pub. Date: Oct. 28, 2010

(54) HYDROGELS FOR COMBINATORIAL DELIVERY OF IMMUNE-MODULATING BIOMOLECULES

(76) Inventors: Ankur Singh, Smyrna, GA (US); Krishendu Roy, Austin, TX (US); Sudhir Kasturi, Atlanta, GA (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 12/764,456

(22) Filed: Apr. 21, 2010

Related U.S. Application Data
(60) Provisional application No. 61/171,663, filed on Apr. 22, 2009.

(51) Int. Cl. A61K 39/29; A61K 38/20; A61K 9/00; A61K 39/00; A61P 31/20

(52) U.S. Cl. 424/484; 424/85.2; 424/227.1; 424/184.1

(57) ABSTRACT

One embodiment of the current disclosure relates to an immune-modulating composition comprising a hydrogel-forming polymer, an immune-modulating biomolecule operable to recruit or retain an immune cell, and an antigen-related biomolecule. Another embodiment of the current disclosure relates to a method of providing an antigen to an antigen presenting cell in an animal by administering to the animal at an administration site an immune-modulating composition as described above. Next, one forms a hydrogel in-situ from the hydrogel-forming polymer, then recruits at least one antigen presenting cell to the administration site using the immune-modulating biomolecule, and finally inducing phagocytosis of the at least one antigen-related biomolecule by the antigen presenting cell.

### CROSS REFERENCE TO RELATED APPLICATIONS

This application claims priority to U.S. Provisional Application No. 61/171,663, filed Apr. 22, 2009, which is incorporated herein by reference.

### STATEMENT OF GOVERNMENT INTEREST

This disclosure was developed at least in part using funding from the National Institutes of Health (NIH R21A1064179-01). The U.S. government has certain rights in the invention.

### BACKGROUND

The immune system protects animals from injury by unwanted foreign organisms, such as viruses, bacteria and parasites. The immune system functions in two basic ways. Innate immunity is a basic protection found even in a very simple animal that simply attacks and kills general types of foreign organisms or foreign materials. Acquired immunity is a much more complicated form of protection in which the body responds to foreign organisms that it has encountered and defended against before.

Acquired immunity forms the basis for many modern medical treatments, particularly acquired treatments like vaccines. If the immune system is first taught to respond to a particular foreign organism or even an aberrant part of the body itself, such as cancer cells, acquired immunity allows the body to attack those unwanted organisms or cells very efficiently. However, in order to prevent the body from learning to attack many harmless things in the environment, acquired immunity requires very particular circumstances before an organism or cell is recognized as dangerous and specifically targeted for destruction.

In one type of acquired immunity, special immune system cells called antigen presenting cells (APCs) have to engulf the unwanted organism or cells, process it into components called antigens, then display those antigens on their surface in special antigen present proteins. Only then can the immune system's attack cells learn to recognize the antigens and attack the unwanted organisms or cells that contain those antigens. This process is further regulated by the need for certain chemicals, often called cytokines (which include chemoattractants and chemokines) to be present for various events to take place. For example, APCs are often found throughout the body and are only recruited specifically to the area where antigens are present by certain chemoattractant chemokines.

Immune-modulating agents, such as vaccines, often fail to cause an acquired immune response, or cause only a weak response, because they do not trigger enough elements of the complicated system used to acquire immunity. For example, it is often useful to use viral DNA as an antigen or to otherwise use DNA to cause the production of antigens in an area where it is injected. Naked DNA vaccines and DNA antigen-loaded microparticles, however, often fail to induce a significant immune response when administered intramuscularly. This is largely due to the fact that significant numbers of APCs are not recruited to the injection site.

One approach to increasing vaccine effectiveness is to co-administer another composition called an adjuvant. The adjuvant is usually something recognized by most immune systems as an unwanted invader. The body therefore begins to fight the adjuvant and in the process looks for new antigens in the area. However, the effectiveness of adjuvants is limited by the fact that the immune system is somewhat engaged in fighting the adjuvant and is not solely focused on the vaccine antigens. Further, many adjuvants trigger such a strong response they cause a great deal of swelling and pain near the injection site and can actually be dangerous to individuals who have a strong immune response to the adjuvant.

Chemokines have also been previously injected with antigens to try to improve vaccination. However, chemokines rapidly leave the administration site and are substantially gone within 24 hours of injection. Although this problem initially seems remediable by repeated injection of chemokines, such daily injections have also proved unsuccessful in at least some studies.

Currently, microparticles have been used to induce an immune response in animals, but without significant success. In particular, microparticles that have been surface functionalized to facilitate uptake by APCs and release from phagosomes in the APCs after uptake have been produced. These microparticles have contained both antigen and chemokines These microparticles have suffered from loss of proteins during formation, inactivation of the proteins after the microparticle is formed, and poor burst release of the proteins. Further, chemokine proteins such as MIP-3 and MCP-1 that need to act on the surface of APCs are useless after the microparticle containing them has been taken up by an APC and cannot help recruit more APCs to the administration site.

### SUMMARY

The present disclosure generally relates to an immune modulating composition and associated methods. More particularly, the present disclosure relates to an immune modulating composition comprising a hydrogel containing at least two different biomolecules and associated methods.

In one embodiment, the present disclosure provides an immune-modulating composition comprising a hydrogel-forming polymer, an immune-modulating biomolecule operable to recruit or retain an immune cell, and an antigen-related biomolecule.

In another embodiment, the present disclosure provides a method of providing an antigen to an antigen presenting cell in an animal by administering to the animal at an administration site an immune-modulating composition as described above. Next, one forms a hydrogel in situ from the hydrogel-forming polymer, then recruits at least one antigen presenting cell to the administration site using the immune-modulating biomolecule, and finally inducing phagocytosis of the at least one antigen-related biomolecule by the antigen presenting cell.

Some embodiments of the disclosure may achieve one or more of the following advantages:
High recruitment of APCs to the administration site;
Co-delivery of multiple (such as two or three or more) immune-modulating biomolecules at the administration site;
Controlled delivery of the immune-modulating biomolecules and antigen-related biomolecules, which may recruit more APCs and allow more effective antigen presentation;
The hydrogel may begin to degrade rapidly after administration to allow an antigen-related biomolecule, including any microparticles, to be present when APCs arrive;
Fine-tuning of the rate of release and degradation of the hydrogel network;
Delivery of large doses of microparticles and chemokines without considerable loss of encapsulation or bioactivity;
Formation of hydrogel at physiological conditions may eliminate the need for photocrosslinking or other conventional crosslinking methods.

One of ordinary skill in the art will recognize that not all embodiments may achieve all advantages and some embodiments may achieve different advantages.

### BRIEF DESCRIPTION OF THE DRAWINGS

A more complete understanding of embodiments of the disclosure will be apparent from the detailed description taken in conjunction with the accompanying drawings which describe various embodiments of the disclosure.

FIGS. 1A and 1B illustrate an overall scheme for preparing a hydrogel and using it to deliver a chemokine and microparticles to an animal.

FIG. 2 illustrates one potential response of dendritic cells to an immune-modulating hydrogel.

FIGS. 3A and 3B illustrate the effects of including Interleukin-10 (IL-10) small interfering RNA (siRNA) in the hydrogel on IL-10 production by APCs.

FIGS. 4A and 4B illustrate the effects on APC cell surface markers by an immune-modulating hydrogel.

FIGS. 5A-5D illustrate the results of in vivo testing for Hepatitis B immunization using an immune-modulating hydrogel.

FIG. 6 illustrates the gel time and composition of various immune-modulating hydrogels.

FIG. 7 illustrates the encapsulation efficiency of various immune-modulating hydrogels.

FIGS. 8A and 8B illustrate the structural morphology of immune-modulating hydrogels.

FIGS. 9A and 9B illustrate release of the chemokine MIP3α from hydrogels with and without microparticles.

FIG. 10 illustrates chemotaxis of APCs in response to different hydrogels with and without microparticles and various chemokine doses.

FIGS. 11A-11C illustrate APC migration studies through collagen in response to an immune-modulating hydrogel.

FIGS. 12A-12F illustrates APC migration through three-dimensional immune-modulating hydrogels.

FIG. 13 illustrates microparticle phagocytosis by APCs in immune-modulating hydrogels.

FIG. 14 illustrates the effectiveness of IL-10 gene silencing in APCs by hydrogel microparticles.

FIGS. 15A-15F illustrates the in vivo Th1/Th2 efficacy of multi-modal delivery of chemokine, siRNA and DNA vaccine delivering hydrogel-microparticle vaccine in a B cell Lymphoma mouse model

FIG. 16 illustrates in vivo cytotoxic T cell activity of multi-modal delivery of chemokine, siRNA and DNA vaccine delivering hydrogel-microparticle vaccine in a B cell Lymphoma mouse model.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are described in more detail below. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The current disclosure relates to immune-modulating compositions and methods of using them. In one embodiment, an immune-modulating composition of the present disclosure comprises a hydrogel-forming polymer, an immune-modulating biomolecule, and a antigen-related biomolecule. In general, immune-modulating biomolecules may recruit or help retain immune cells, such as antigen presenting cells (APC) in the area where the immune-modulating biomolecule is located. Additionally, antigen-related biomolecules may induce a specific response, such as an antigen specific response, in the recruited immune cells. The immune-modulating composition may contain multiple different molecules of each type. Other types of biomolecules, such as biomolecules able to increase antigen presentation or the efficiency of attack cell reaction to the presented antigen, may also be included. In one embodiment, the antigen-related biomolecule may be in a microparticle to modulate the timing of its release.

Immune-modulating biomolecules suitable for use in the present disclosure may be a cytokine, such as a chemokine or a chemoattractant. For example, in some embodiments, it may be a chemokine able to attract and/or retain APCs. Target APCs may include any APC involved in inducing an acquired immune response, particularly an acquired immune response to the antigen-related biomolecule. Specific APCs that may be targeted include Langerhans cells and dendritic cells, such as myeloid dendritic cells. In some embodiments, immature APCs may be targeted for recruitment. Examples of suitable chemokines may include, but are not limited to, Macrophage Inflammatory Protein 3α (MIP3α), Monocyte Chemotactic Protein-1 (MCP-1), MIP1α, MIP1β, Secondary Lymphoid Tissue Chemokine (SLC), N-formyl-methionyl-leucyl-phenylalanine (fMLP), IL-8, Regulated on Activation Normal T Cell Expressed and Secreted (RANTES, also known as Chemokine (C—C motif) Ligand 5 or CCL5), and stromal cell-derived factor-1 (SDF-1), or any combinations of these and other factors. In specific embodiments, an immune-modulating composition of the present may contain two or more or three or more types of immune-modulating biomolecules.

In a specific embodiment, an immune-modulating biomolecule may be a molecule, such as a protein or peptide, that is normally rapidly removed when injected into a tissue, for example by diffusion or degradation. The hydrogel may slow this movement of the biomolecule or release more of it over time to allow for a longer period during which the amount of the immune-modulating biomolecule is elevated near the administration site. For the example, the hydrogel may release any immune-modulating biomolecule in such a manner as to create a sustained gradient over a few days. This sustained gradient may increase both the number of immature APCs at the administration site and/or the duration of their presence.

Antigen-related biomolecules suitable for use in the present disclosure may be any type of biomolecule linked to an agent that ultimately triggers an immune response. For example, it might be the agent itself or something that metabolizes the agent or causes it to be produced. In examples where the agent is an antigen, the second type of biomolecule may be the antigen, a nucleic acid containing the antigen, or a protein or other molecule cleaved or modified in an antigen presenting cell to produce the antigen. In particular embodiments, the second type of biomolecule may be an antigen able to induce a vaccinating immune response, such as any currently used vaccine antigens. The second type of biomolecule may also be an antigen derived from a cancer cell.

In some embodiments, an antigen-related biomolecule may be included in a microparticle. Microparticles may improve uptake of an antigen-related biomolecule because they are often readily taken up by APCs. The synthetic nature of microparticles as well as their size (microns), which is similar to that of many pathogens, may facilitate this uptake by APCs. In some embodiments, microparticles suitable for use in the present disclosure may be cationic in order to enhance delivery of their cargos to the cytoplasm by buffering the phagosomes in which they end up after being taken up by the APCs. Microparticles may also persist at the administration site longer when present in a hydrogel than if simply injected or otherwise administered.

Microparticles in certain embodiments may be made from synthetic polymers like polyesters, polyanhydrides, polycaprolactone, natural polymers like hyaluronic acid, chitosan, alginate, dextran, as well as lipid based materials like phosphatidyl choline, and the like.

In some embodiments, one or more different types of antigen-related biomolecules may be included in an immune-modulating composition of the present disclosure. For example, in one embodiment, two different types of nucleic acids may be included. In some embodiments where microparticles are used, the one or more different antigen-related biomolecules may be both included in the same microparticle or they may be in separate microparticles. There may be advantages to including both in one microparticle so that an APC need to potentially only take up one microparticle to present the antigen.

Additional biomolecules that are neither immune-modulating biomolecules nor antigen-related biomolecules may also be included in an immune-modulating composition of the present disclosure. For example, chemokines that recruit attack immune cells or facilitate their recognition of antigens on APCs may be included. siRNA that downregulates various proteins in the APCs may also be included as this downregulation facilitates the overall desired immune response. Similarly, plasmids or other nucleic acids encoding proteins or peptides that facilitate the overall desired immune response may also be included. For example, the production of IL-10 by APCs may be decreased or increased to induce either a TH-1 type immune response (more effective against intracellular pathogens such as viruses) or a TH-2 type immune response (more effective against extracellular pathogens such as most bacteria).

These additional biomolecules may be included in an immune-modulating composition alone or they may also be part of any microparticles. The most appropriate location for any additional biomolecules may be determined by when they need to be released and where they need to go to be effective. If the additional biomolecules need to cause a particular effect within the APCs, inclusion in microparticles may be more effective. As in the case of different examples of the antigen-related biomolecules, the additional biomolecules may be in separate microparticles or combined in microparticles with other molecules.

As mentioned previously, an immune-modulating composition of the present disclosure comprises a hydrogel forming polymer. In some embodiments, a hydrogel forming polymer may crosslink once administered and form a hydrogel only after an additional ingredient is added or conditions are altered to match administration-site conditions, such as temperature or pH. Use of a hydrogel forming polymer may facilitate administration of the hydrogel. Hydrogels formed after administration may be referred to as in-situ crosslinkable hydrogels. In example embodiments, the hydrogel may be subject to hydrolytic degradation under physiological conditions normally present at the administration site. The hydrogel may also be made of biocompatible materials such as a biocompatible polymer.

In specific embodiments using in-situ crosslinkable hydrogels, particularly those administered by intramuscular injection, a hydrogel forming polymer suitable for use may include, but are not limited to, a vinyl sulfone, an acryl-derivatized polysaccharide, a thiol-derivatized polysaccharide, an acryl-derivatized polyethyleneglycol, a thiol-derivatized polyethyleneglycol, and any a combination thereof. In situ polymerization may allow a high loading capacity of an immune modulating biomolecule and may allow more than one different type to be used.

The chemical composition, polymer concentration, degree of crosslinking and other properties of a hydrogel of the present disclosure may be varied to influence the rate of degradation and thus the rate of release for various components. In specific embodiments, an immune-modulating composition of the present disclosure may be injected into a patient and form a hydrogel within about forty to sixty seconds after injection. Longer times for hydrogel crosslinking may also be suitable, so long as inappropriate amounts of biomolecules are not lost prior to hydrogel formation.

According to one very particular embodiment, an immune-modulating composition may comprise a hydrogel forming polymer capable of crosslinking in-situ and comprising chemokines to attract immature dendritic cells, such as MIP3α, as well as antigen-loaded microparticles.

In another embodiment, a hydrogel may be formed prior to administration or it may be administered in an uncrosslinked form. If administered in an uncrosslinked form, it may then crosslink at physiological temperature and pH. In various examples, the crosslinked or uncrosslinked hydrogel may be administered intramuscularly, subcutaneously, or intradermally.

After crosslinking, any immune-modulating biomolecule, such as a chemokine, may be released from the hydrogel in a sustained fashion to recruit and/or retain APCs at the site of administration. These APCs may then take up (phagocytose) an antigen-related biomolecule, which may be a microparticle, ultimately triggering the presentation of antigens by the APCs and the recognition of those antigens by immune system attack cells. The antigen-related biomolecules may become more available as the hydrogel degrades, for example over the course of three to four days. The APCs may take up the antigen-related biomolecule when it is released from the hydrogel, after entering the hydrogel, or both.

In particular embodiments, the hydrogel may degrade over three to four days, allowing synchronization between APC recruitment and availability of the antigen-related biomolecule.

The immune-modulating compositions and related methods of this disclosure may be used for a variety of purposes. For example, they may be used as vaccines or for delivering multiple growth factors to the body at different release rates. Overall the immune-modulating compositions may allow ready substitution of specific co-administered agents. For example, any chemokine may be used depending on the immunological requirements of the system such as the administration site, the antigen, and the target immune cells. The immune-modulating compositions may also be used for multi-modal delivery of various biomolecule such as CpG oligos, interleukins, various proteins and the like.

### EXAMPLES

The present disclosure may be better understood through reference to the following examples. These examples are included to describe exemplary embodiments only and should not be interpreted to encompass the entire breadth of the invention.

### Example 1

### Outline of Overall Proposed Delivery System Design

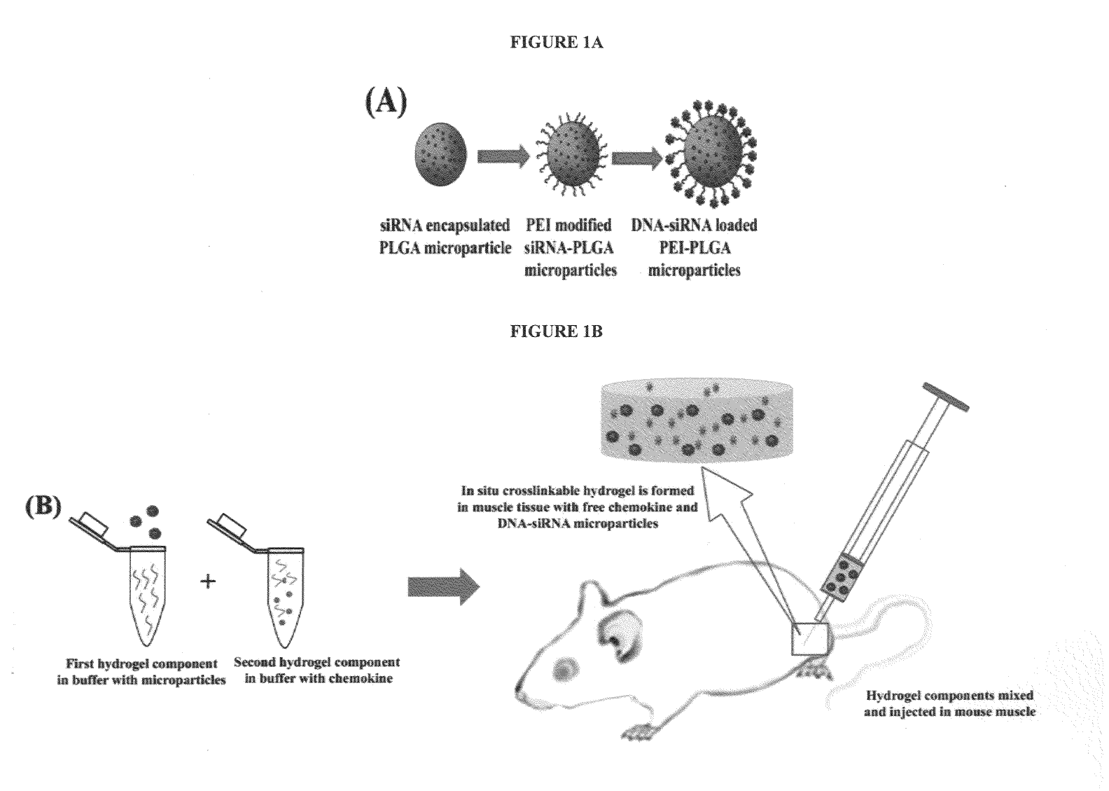

As illustrated in FIG. 1A, microparticles containing antigen DNA and other immune-modulating biomolecules may be prepared. First siRNA, such as IL-10 siRNA may be encapsulated in a poly(lactic-co-glycolic acid) (PLGA) microparticle. This microparticle may then be modified with polyethyleneimine (PEI) to make them cationic. Next, antigen DNA, for example in the form of a plasmid, may be electrostatically loaded onto the microparticles.

As illustrated in FIG. 1B, the DNA and siRNA co-loaded microparticles may be mixed in a first hydrogel component then a chemokine, such as MIP3α may be mixed in a second hydrogel component.

Finally, as illustrated in FIG. 1C, the two solutions may be mixed together and administered, for example by intramuscular injection into an animal, where they form an in situ crosslinked hydrogel with the chemokine and microparticles entrapped in it.

### Example 2

### Dendritic Cell Trafficking

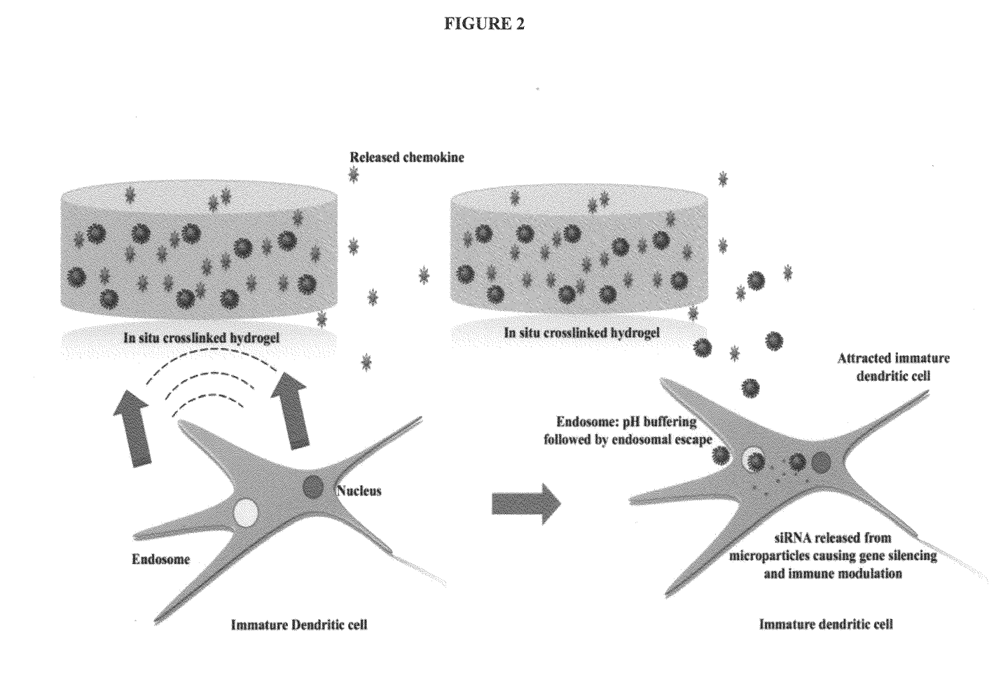

As illustrated in FIG. 2, the in-situ crosslinked hydrogel releases the chemokine first which attracts naive (immature) dendritic cells (DCs) to the vicinity. The DNA/siRNA carrying microspheres are later released (or the DCs enter the hydrogel) as the hydrogel degrades and are phagocytosed by DCs. The presence of secondary or tertiary amines on the particle surface increases its buffering capacity so it is able to escape from the endosome (phagosome) after phagocytosis and enter the cytoplasm of the DC. In the cytoplasm, the microspheres release siRNA, which silences a corresponding gene, and allows the DNA to be delivered to the nucleus of the same cell to later cause antigen presentation.

### Example 3

### Immune-Modulation by Combinatorial, Single Formulation Delivery of siRNA and a DNA Plasmid—pDNA Expression and siRNA Effectiveness

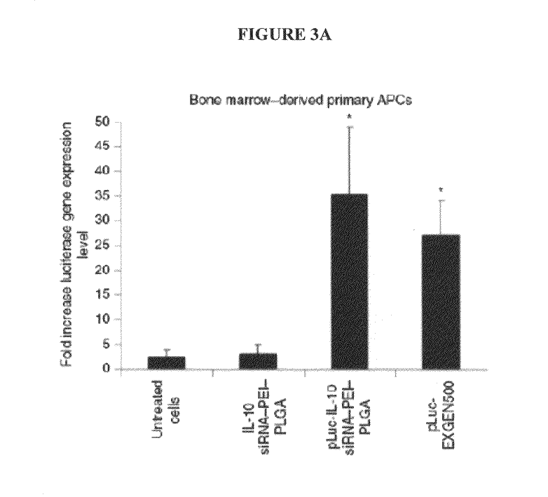

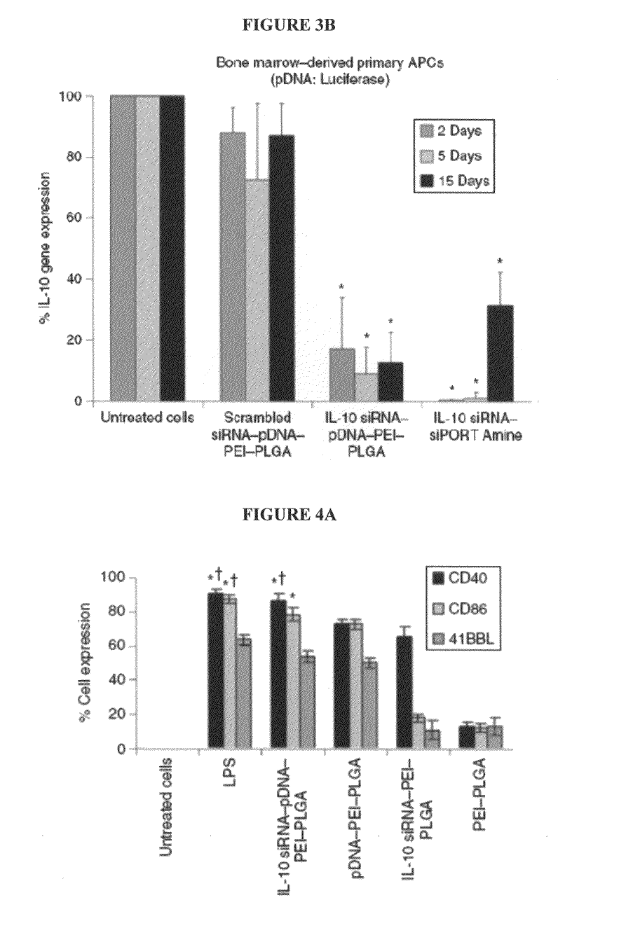

As shown in FIG. 3, microparticles were prepared as in Example 1 IL-10 siRNA and plasmid DNA (pDNA) encoding Luciferase. Bone marrow-derived primary APCs were transfected with these microparticles. The microparticle showed a greater increase in Luciferase expression than was even achieved with the traditional EXGEN500 transfection system. No significant increase in Luciferase expression was seen in untreated cells or cells treated with microparticles lacking the pDNA. (FIG. 3g). Thus, the microparticles were effective at delivering the pDNA to APCs in a fashion that allowed its expression by the cells.

Similarly, although less effective two and five days after transfection, the microparticles were nevertheless similarly effective in decreasing IL-10 expression as IL-10 siRNA delivered using the traditional siPORT Amine system. Significant decreases in IL-10 expression were not seen in untreated cells or cells treated with microspheres containing scrambled siRNA. (FIG. 3h). Accordingly, the microparticles were also effective at delivering siRNA to the APCs in a fashion able to silence IL-10 RNA for at least fifteen days.

### Example 4

### Immune-Modulation by Combinatorial, Single Formulation Delivery of siRNA and a DNA Plasmid—Cell Surface Marker Effects

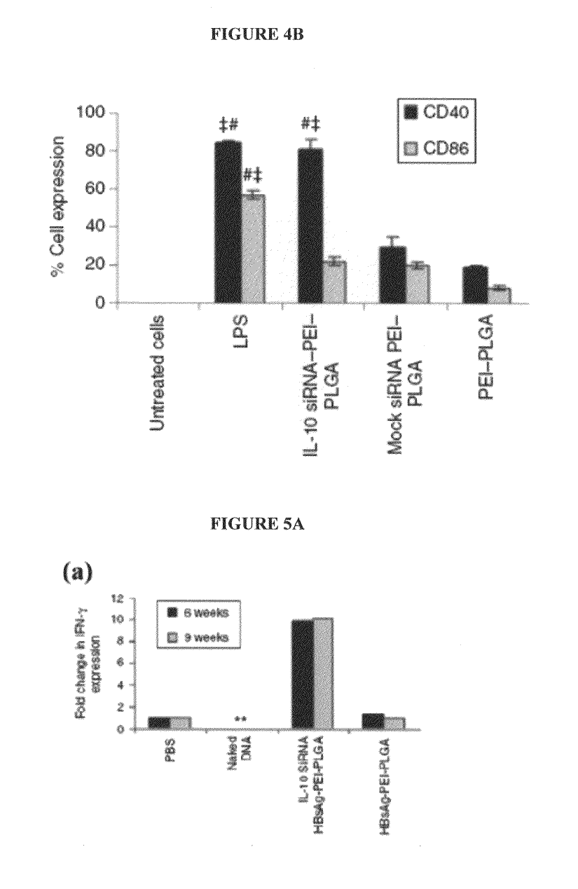

FIG. 4 illustrates the effects of the microparticles from Example 3 on cell surface markers in transfected APCs. The presence of these particular cell surface markers indicates the maturation of APCs and thus is a classic indicator or APC activation, which is required for a strong immune response. Therefore, high expression levels of the markers on APCs transfected with the microparticles of Example 3 demonstrates that they are mature and able to cause a strong immune response.

### Example 5

### Immune-Modulation by Combinatorial, Single Formulation Delivery of siRNA and a DNA Vaccine

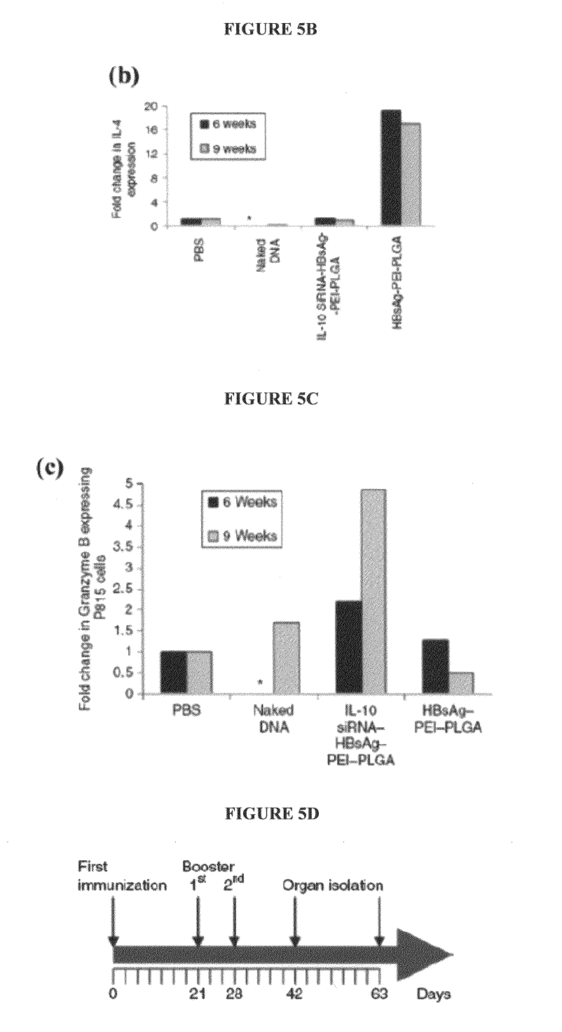

FIG. 5 illustrates the general timeline for administration of a microparticle to mice in order to study Hepatitis B DNA expression. The microparticle was prepared in the same manner as the microparticle in Example 3, but the pDNA was a plasmid encoding Hepatitis B Surface Antigen (HbsAg) instead of Luciferase. At both six and nine weeks after first immunization, Interferon-γ (IFN-γ) expression was much higher in mice that received the microparticle than in mice that received a microparticle with no siRNA, naked plasmid DNA, or phosphate buffered saline (PBS). (FIG. 5A). This shows that the DNA/siRNA microparticle alone induced a significant anti-viral immune response in the mice.

IL-4 levels were increased in mice that received microparticles lacking the siRNA, but were low in mice receiving the DNA/siRNA microparticles, naked DNA, or PBS. This indicates a significant divergence towards TH1 type immune response (as indicated by high IFNγ levels and low IL-4 levels) thereby confirming the immuno-modulatory effect of the formulation. (FIG. 5B).

### Example 6

### In Situ Crosslinkable Hydrogels—Gelling Properties and Microparticles

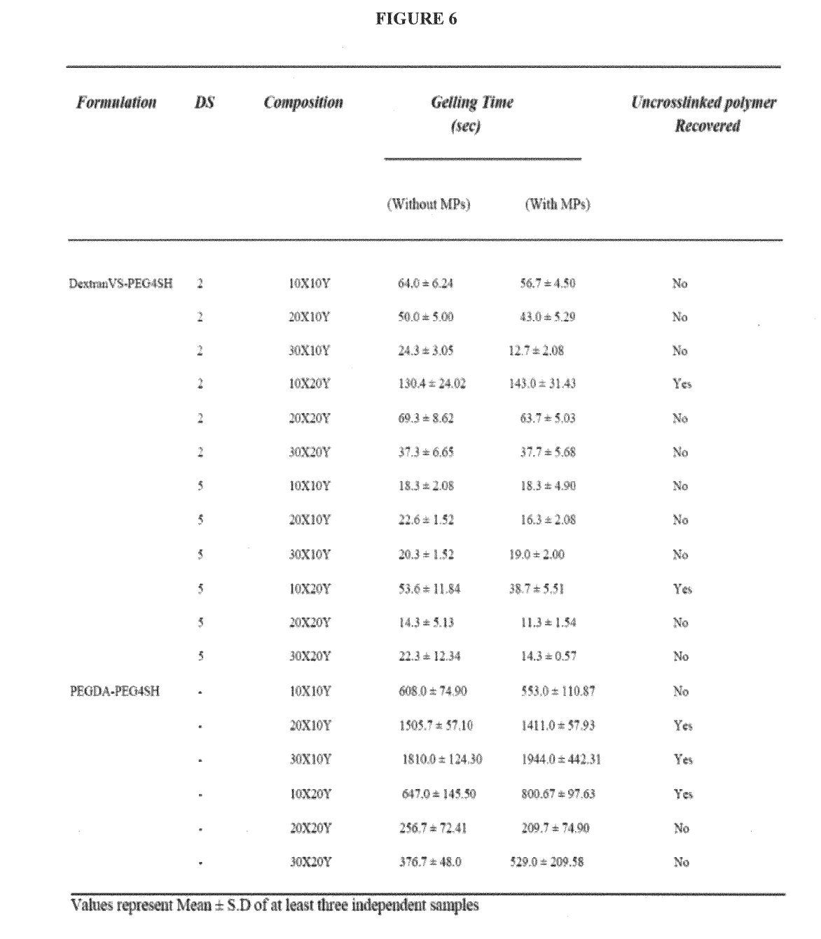

FIG. 6 provides gelation time and other information for in-situ crosslinkable hydrogels. Some hydrogels are made primarily of Dextran Vinyl Sulfone crosslinked to tetra-thiolated polyethylene glycol (Dextran VS-PEG4SH) or polyethyleneglycol diactrylate crosslinked to tetra-thiolated polyethylene glycol (PEGDA-PEG4SH). “DS” designates the degree of substitution of the hydrogel polymers. X designates the weight/volume (w/v) percentage of the first hydrogel components (e.g. Dextran VS or PEGDA) and Y represents the w/v percentage of the second hydrogel component (e.g. PEG4SH). A 100 μL hydrogel was prepared in each example. Gelling time with and without microparticles and the presence of any unlinked polymer is also reported. Thus, adequate gelling even in the presence of microparticles may be obtained with a variety of different hydrogel compositions.

Synthesis of dextran vinylsulfone (DextranVS) with ethyl spacer was performed as mentioned earlier using N,N′-dicyclohexyl-carbodiimide and 4-(Dimethylamino)pyridinium 4-toluenesulfonate (DPTS) catalyst. DPTS was synthesized by dissolving 5 g of pTSA monohydrate in 100 ml Tetrahydrofuran (THF). 4-(dimethylamino)-pyridine (DMAP, 99%) at one molar equivalent to pTSA was added to this mixture and filtered to obtain precipitate which was further dissolved in dichloromethane and recrystallized using a rotary vacuum evaporator. Dextran vinyl sulfone ester synthesis was performed by adding 16.425 g DVS in 90 ml of inert nitrogen saturated DMSO followed by drop wise addition of 0.75 g 3-MPA to it under continuous stirring (molar ratio of 3-MPA to DVS was 1:20). The reaction was continued for 4 hrs at room temperature in the dark. The reaction was performed in the dark to avoid any photo-crosslinking of vinyl sulfone moieties. 5 g or 2.5 g Dextran was dissolved in 30 ml DMSO and solution of 2.17 g DCC and 0.32 g DPTS in 30 ml DMSO was added to it drop-wise and stirred until clear solution was obtained. DPTS is a weak acidic catalyst and enhances the reaction efficacy of DCC. Finally, the mixture was added to DVS/MPA solution in the dark and the reaction was allowed to proceed for 24 hrs at room temperature. After the completion of reaction, N,N-dicyclohexylurea (DCU) salt was filtered using a vacuum filter and the product was recovered by precipitation in 1000 ml of ice cold 100% ethanol. The precipitate was separated from residual ethanol through centrifugation at 3000 rpm for 15 min followed by vacuum drying. Precipitate was re-dissolved in at least 100 ml of de-ionized water (pH adjusted to 7.8) and vortexed to obtain a clear solution. Finally, unreacted polymer was removed through ultra filtration using Amicon filter (MWCO 10000 Da, Millipore) and the viscous product was lyophilized to remove water, analyzed using NMR.

### Example 7

### In Situ Crosslinkable Hydrogels—Chemokines and Encapsulation Efficiency

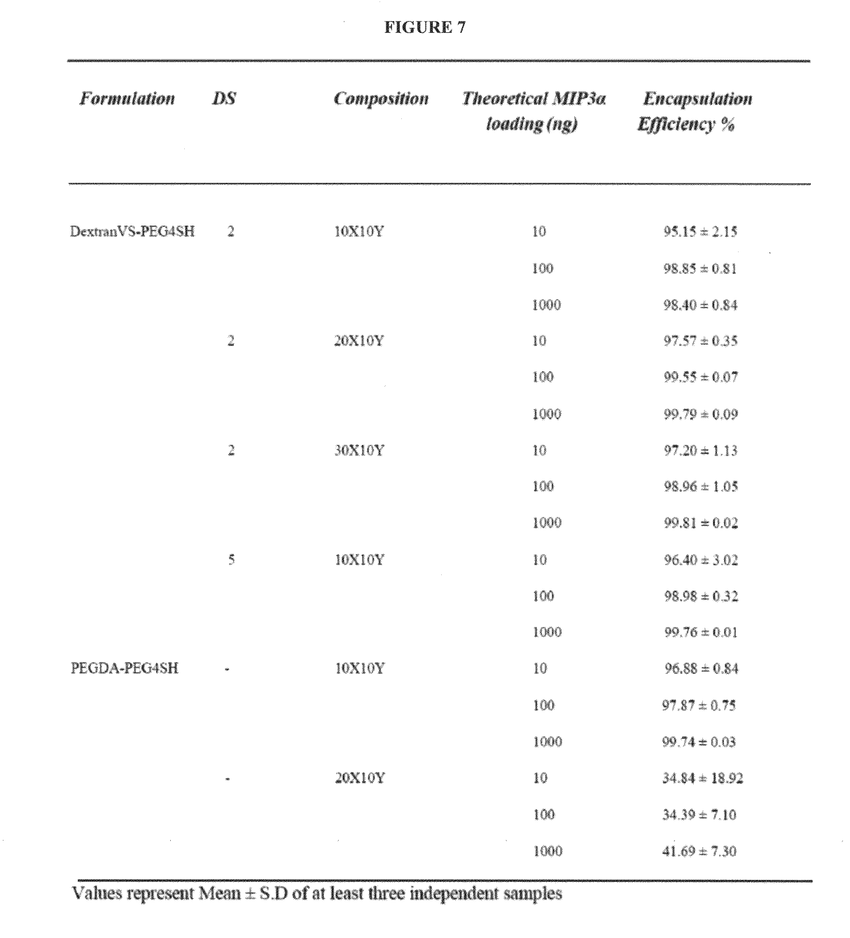

FIG. 7 presents the theoretical chemokine loading efficiency for MIP3α. “Theoretical” denotes the initial loading attempted, i.e. 100% of the starting amount of chemokine FIG. 7 also presents the efficiency with which the chemokine is encapsulated (i.e. the proportion of the theoretical amount actually encapsulated) for hydrogels described in Example 6.

### Example 8

### Structural Morphology of Hydrogels With and Without Microparticles

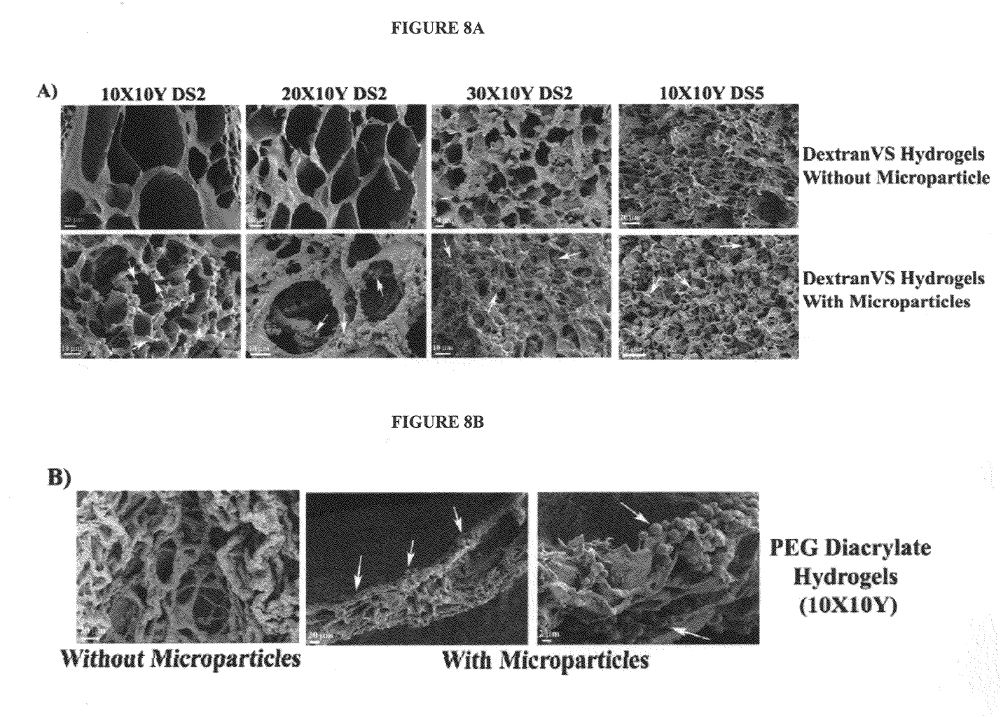

FIG. 8 shows scanning electron microscope (SEM) images of various hydrogels having compositions described in Examples 6 and 7. Hydrogels of both types tested with different relative amounts of the hydrogel components are shown. Microparticles are labeled with arrows. As the images show, all tested hydrogels were able to incorporate microparticles into their structure. The microparticles tended to be deposited in layers.

### Example 9

### In Vitro Release Studies From Hydrogels

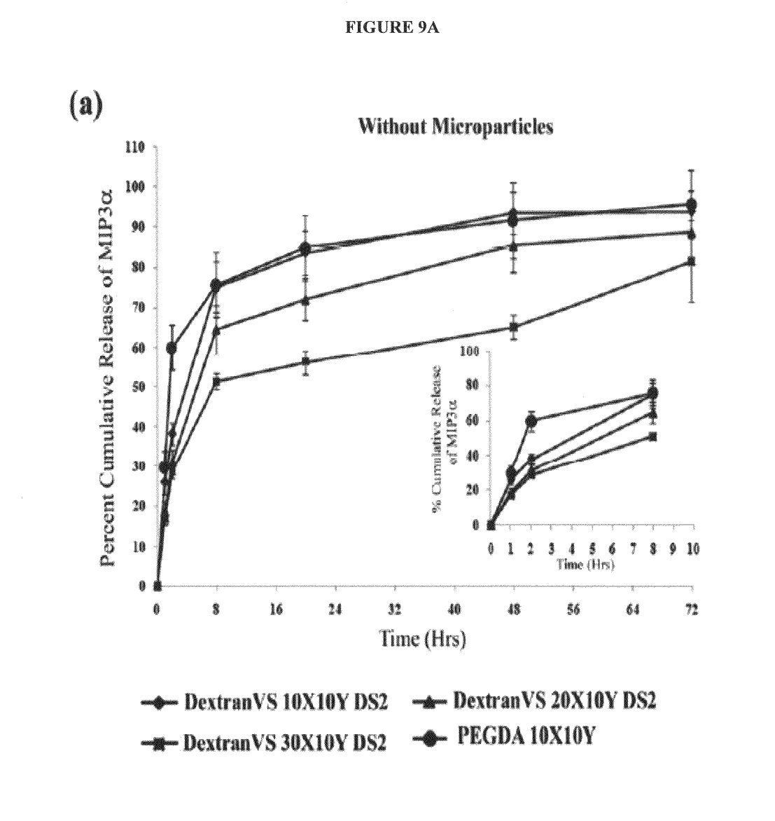

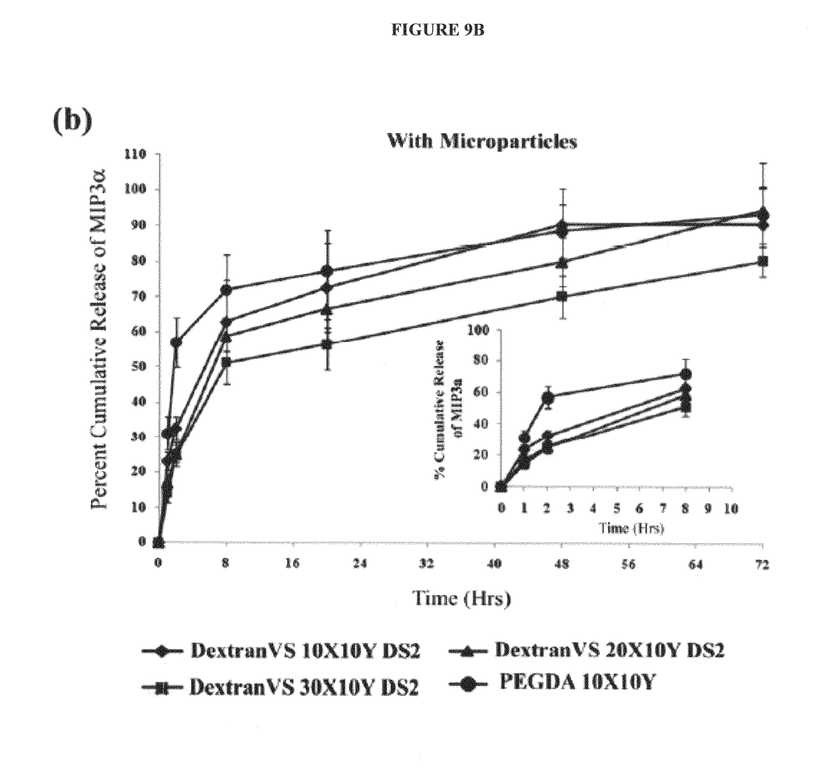

FIG. 9 presents in vitro MIP3α release data for various hydrogels of the types described in Examples 6-8. Release of MIP3α is rapid, reaching a high level after only hours, then plateaus for steady release. This rapid initial release followed by steady levels should allow the MIP3α to recruit APCs to the administration site quickly, then continue to recruit new APCs for several days.

### Example 10

### In Vitro Chemotaxis

FIG. 10 presents in vitro chemotaxis data gathered using the Transwell™ Chemotaxis Protocol. Hydrogels of the type described in Example 6-8 were loaded with 50 ng of MIP3α per 100 uL of gel. Hydrogels were prepared with different polymer concentrations. Similar amounts of chemotaxis were observed with different gels with or without microparticles, showing that the regular release of MIP3α observed in Example 10 is effective a recruiting APCs.

### Example 11

### APC Migration Through Collagen Tissue Model

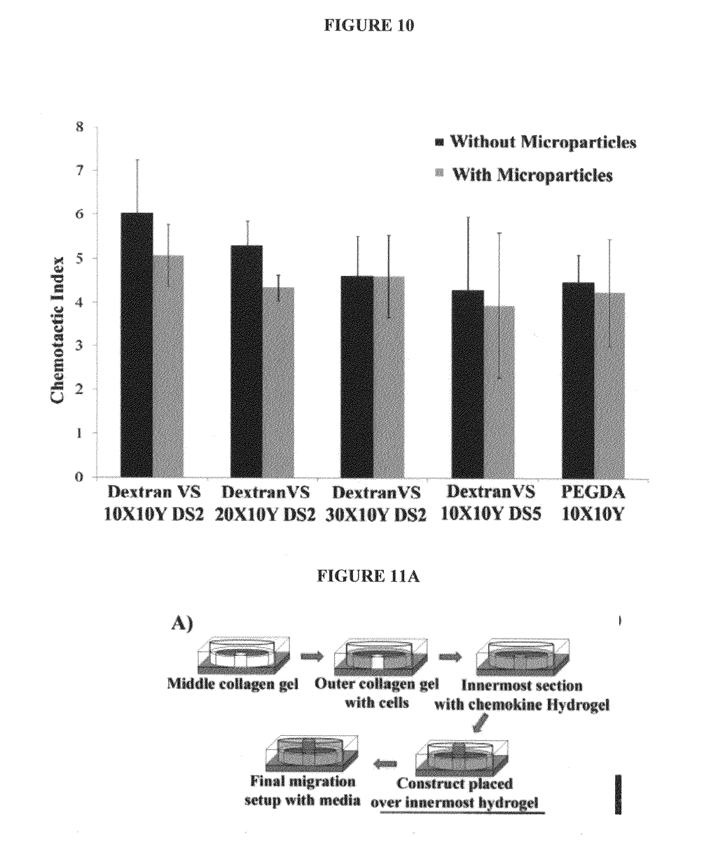

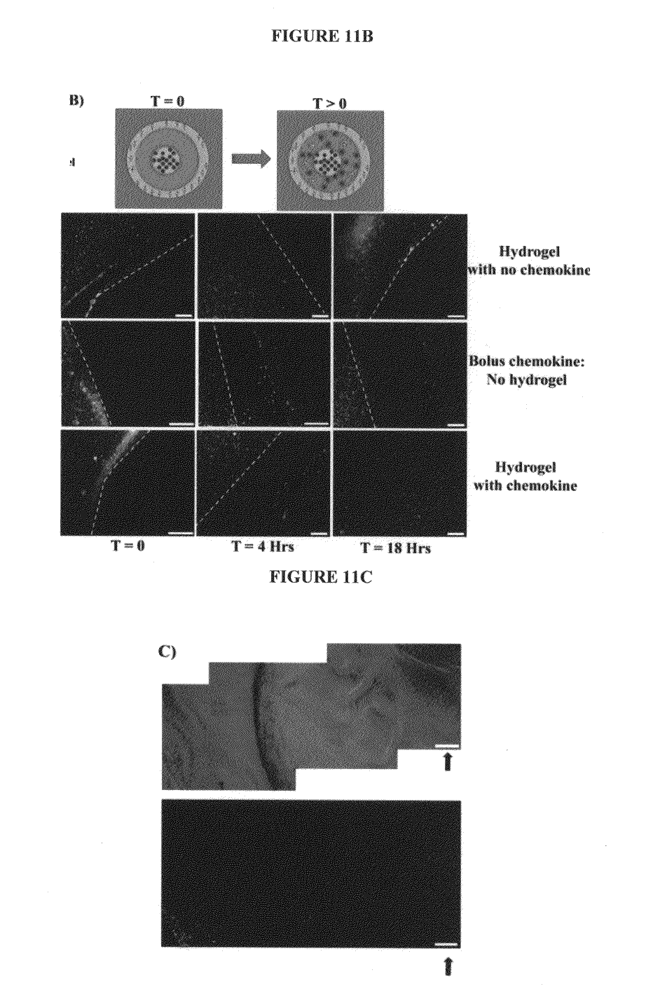

FIG. 11A shows a schematic representation of how a collagen gels was prepared and used to test whether various hydrogels of the current disclosure could cause APC to migrate through the gel. The ability of APCs to migrate through a collagen gel correlates with the ability of APCs to migrate through living tissue. To prepare the collagen gels, each well of a six well place was sectioned into three concentric zones using polydimethylsiloxane (PDMS) molds. The middle zone was filled with a collagen solution and allowed to gel for 30 minutes. The outermost zone was filled with primary APCs and red polystyrene particle-containing collagen solution and allowed to gel for 30 minutes. The innermost zone was filled with the Dextran VS DS2 10X10Y (or a bolus of MIP3α chemokine for the relevant control) and allowed to gel for 30 minutes. A metallic construct was placed on top of the innermost zone and culture media was added to the outer zones.

FIG. 11B shows how Primary APCs initially present in the outermost zone migrated into the middle and inner zones. Chemokine also migrated into the middle zone. In the panel photographs, one can see the APC migration after 4 hours and 18 hours when either the chemokine bolus or the hydrogel were present in the middle zone, but not when no chemokine was used. (White broken lines show the initial APC zone-collagen zone boundary at time zero.) Thus, the hydrogels of the current disclosure are able to cause APC migration into collagen and are expected to be able to cause similar migration in living tissue.

FIG. 11C shows APCs migrating into the center hydrogel in response to the chemokine The top panel shows a phase contract image of sequential frames attached together to form a single image. APC migration can be seen as small circular cells moved or accumulated towards the right of the image. The bottom panel shows a fluorescent image of sequential frames attached together to form a single image. Polystyrene particles are red, Calcein-stained primary APCs are green. Movement of APCs into the collagen zone and hydrogel can be seen. This shows that APCs can move into hydrogels of the current disclosure where the microparticles are also located.

### Example 12

### APC Infiltration of Hydrogels

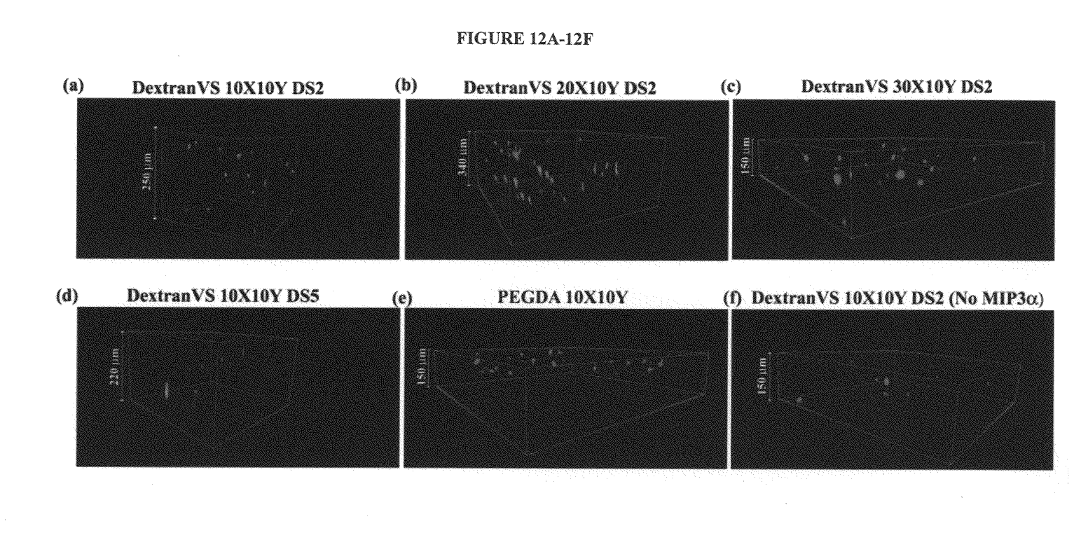

FIG. 12 shows migration of APCs through hydrogels. Various hydrogels of the type described in Example 6-8 as well as a control hydrogel lacking chemokine were prepared with red-labeled microparticles. Green-labeled APCs were placed in proximity to the hydrogels to study whether the APCs would enter the hydrogels. All hydrogels with chemokine showed substantial infiltration while the hydrogel lacking chemokine did not. Accordingly, the hydrogel infiltration is responsive to the chemokine. This data also further confirms the ability of APCs to enter a variety of hydrogels according to the current disclosure.

### Example 13

### Phagocytosis of Microparticles

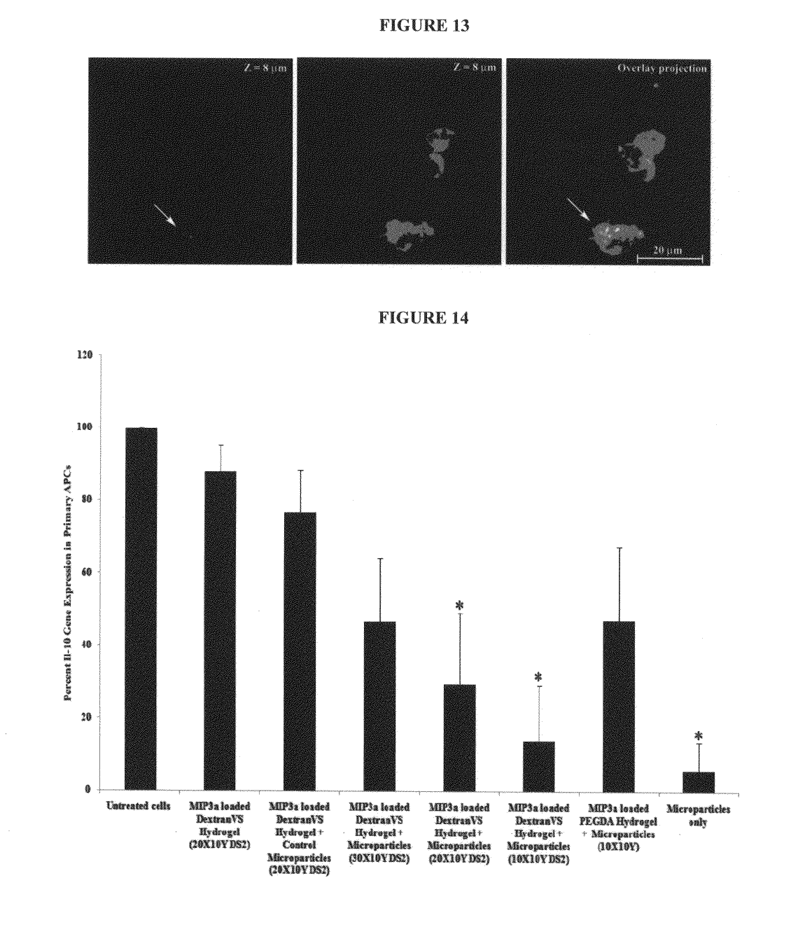

FIG. 13 shows various green-labeled APCs inside a hydrogel of the current disclosure. Microparticles are labeled red. An overlay of the red (first) and green (second) images in the third panel clearly shows that red particles are located inside the APCs, indicating that they were taken up by those APCs via phagocytosis.

### Example 14

### In Vitro Gene Silencing Efficacy of Microparticles in Hydrogels

FIG. 14 present data showing IL-10 gene silencing in APCs that have taken up microparticles from hydrogels of the current disclosure. Bone marrow-derived primary APCs were tested 5 days after exposure to hydrogel-embedded PEI-PLGA microparticles containing pgWizLuciferase pDNA and IL-10 siRNA. IL-10 gene silencing was quantified using RT-PCR. All test groups were subjected to a hydrogel-based formulation of they type described in Example 6-8 except the untreated and microparticles only groups. Control nanoparticles contained a scrambled siRNA sequence. Hydrogels containing no microparticles or only control microparticles showed little decrease in IL-10 expression as compared to untreated cells. All hydrogels containing functional control particles, showed much more significant decreases. One hydrogel, Dextran VS DS2 10X10Y showed almost as much decrease in IL-10 expression as when cells were transfected with control microparticles. Overall, the results indicate that microparticles contained in hydrogels of the current disclosure are taken up by APCs and siRNA in the microparticles is able to disrupt protein expression of immune-modulation proteins within the APCs.

### Example 15

### In Vivo Immune-Modulation in a Weakly Immunogenic A20 B Cell Lymphoma Mouse Model: Proof of Concept

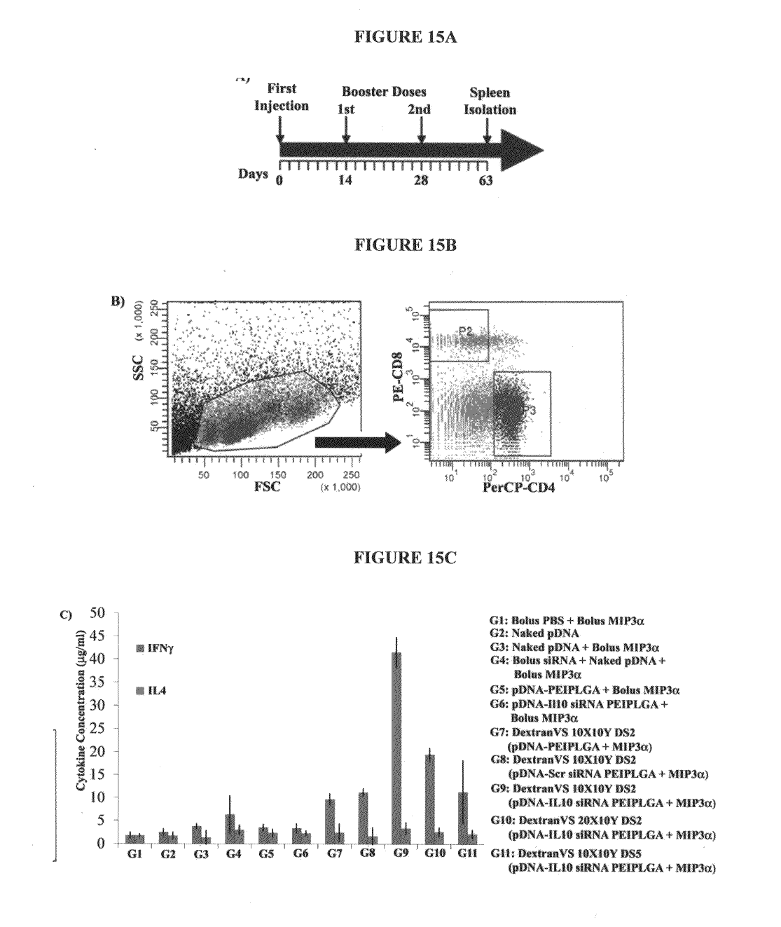

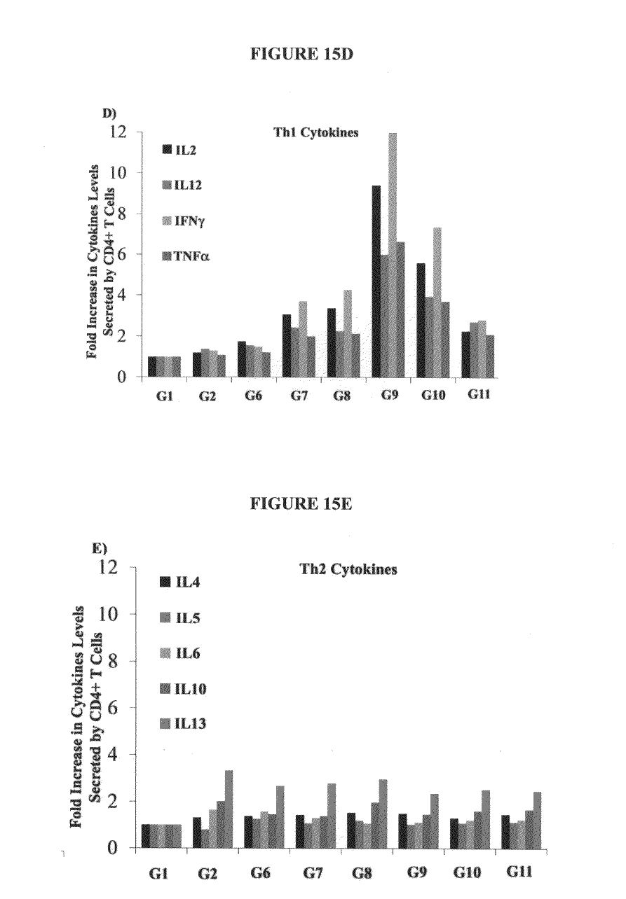

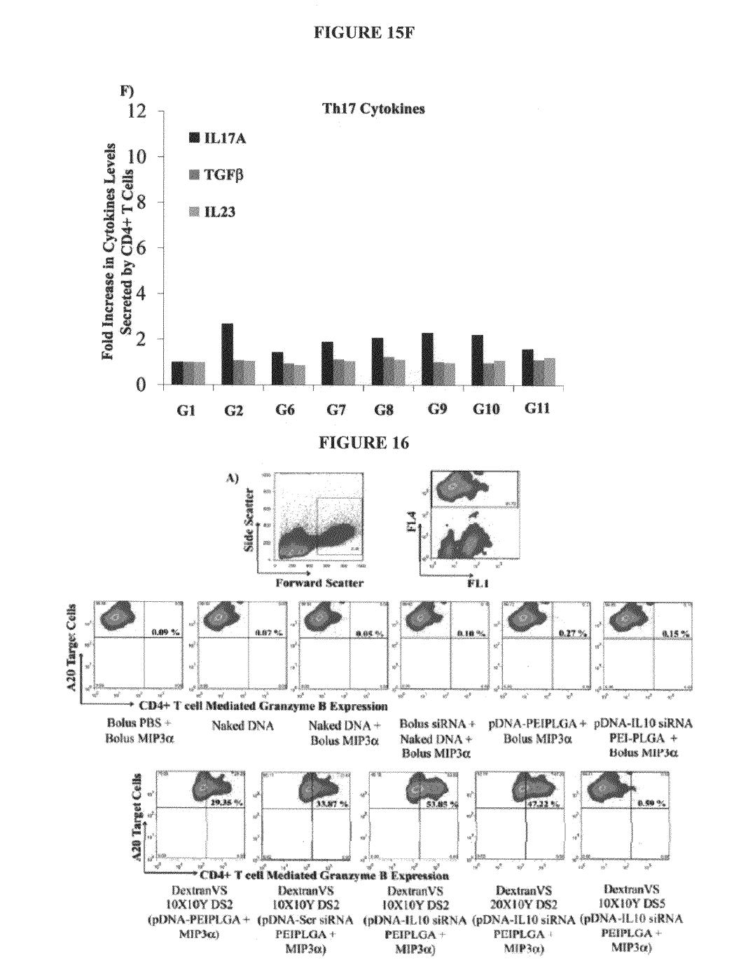

To systematically study the immune response arising from various formulations, microparticle with or without encapsulated IL10 siRNA were administered intramuscularly in Balb/c mice (FIG. 15A). Further, to evaluate the effect of creating immune priming center, as well as explore the effect of degradation rate of hydrogel on extent and type of immune response, mice were immunized with an immune modulating composition of the present disclosure. These compositions contained MIP3α and DNA/siRNA microparticles. Pooled splenocytes from 5 mice per group were purified into CD4+ and CD8+ cells using flow cytometry (FIG. 15B) and re-stimulated with naïve splenocytes incubated with A20 protein. FIG. 15C shows concentrations of CD4+ cells released Th1 specific IFN-γ and Th2 specific IL4 in the culture medium. IFN-γ production increased markedly when fast degrading DextranVS 10X10Y DS2 hydrogels were used to co deliver chemokine and DNA-IL10 siRNA loaded microparticles. IL4 expression was markedly restricted in all formulations, strongly suggesting that a “skewed” CD4+ T helper cell response towards Th1 phenotype in mice immunized with 10X10Y DS2 hydrogels. Bolus chemokine and pDNA loaded microparticles immunized animals or in fact, co-delivering IL10 siRNA failed to induce immune response in this weak immunogenic tumor model. However, when chemokines and microparticles were delivered using fast degrading hydrogels, an increase in IFNγ levels was observed with no change in IL4. It is also clear that the IFNγ level was maximum for the group that received 10X10Y DS2 hydrogels with chemokine and DNA-L10 siRNA microparticles as compared to 10X10Y DS2 hydrogels with chemokine and DNA-scrambled siRNA microparticles. This IFNγ and IL4 ELISA was used to screen and select samples from 11 groups for further analysis of various Th1/Th2/Th17cytokines. As shown in FIG. 15D, levels of Th1 specific cytokines IL2, IL12, and TNFα were ˜10, 6 and 7 fold higher respectively than PBS immunized animals. Naked DNA or DNA-IL10 siRNA microparticles immunized animals showed 1-2 fold increase only while slow degrading DS5 hydrogel group showed only 2-3 fold increase. Between the same DS2 hydrogels, 20X10Y hydrogels showed the intermediate response between 10X10Y DS2 and DS5 formulations. The IL2 levels in 20X10Y immunized animals were 6 fold while IL12 and TNFα were ˜4 folds higher than PBS immunized animals. Levels of IL2, IL12, and TNFα in 10X10Y DS2 hydrogels with chemokine and only DNA microparticles or scrambled siRNA loaded microparticles were between 2-4 folds higher than PBS immunized animals. Levels of Th2 cytokines including IL5, IL6, IL10, and IL13 were markedly low across all the groups (FIG. 15E). Thus, consistent low levels of Th2 cytokines and an increase in Th1 specific cytokines with DextranVS 10X10Y DS2 hydrogels indicate a strong shift towards Th1 type immunity even with a weakly immunogenic A20 idiotype DNA vaccine. Levels of Th17 cytokines were also markedly low with IL23 and TGF13 levels comparable to PBS across all groups. IL17 cytokines were 1.5-3 folds higher than PBS however no strong difference was observed between any formulations treated groups (FIG. 15F).

Granzyme B levels in target cells were measured to assess the CTL activity by T cells. Purified CD4+ and CD8+ T cells were co-incubated with A20 murine B cell Lymphoma tumor cells at 20:1 E:T ratio for two hours. Flow cytometry based measurement of Granzyme B activity inside target cells provides an early time point sensitive, quantitative assessment of T cell-mediated cytotoxicity. The results indicated that CD4+ cell mediated Granzyme B response was essentially low in mice immunized with naked DNA or microparticles delivering DNA with or without IL10 siRNA. Even bolus supplement of chemokine failed to boost up any CTL response. On the other hand, fast degrading DS2 hydrogels showed stronger Granzyme B positive response as indicated in the double positive quadrants of each plot (FIG. 16). Strikingly, with inclusion of IL10 siRNA, the response was stronger (53.6% versus 29.12%) %). DS5 hydrogels did not show any marked CTL activity.

Although the present disclosure has been described in several embodiments, a myriad of changes, variations, alterations, transformations, and modifications may be suggested to one skilled in the art, and it is intended that the present disclosure encompass such changes, variations, alterations, transformations, and modifications as falling within the spirit and scope of the appended claims.

1. An immune-modulating composition comprising:
at least one hydrogel-forming polymer;
at least one immune-modulating biomolecule operable to recruit or retain an immune cell; and
at least one antigen-related biomolecule.

2. The composition of claim 1, wherein the at least one hydrogel-forming polymer comprises as hydrogel precursor operable to form a hydrogel under physiological conditions in an animal.

3. The composition of claim 1, wherein the hydrogel-forming polymer is selected from the group consisting of: a vinyl sulfone, an acryl-derivatized polysaccharide, a thiol-derivatized polysaccharide, an acryl-derivatized polyethyleneglycol, a thiol-derivatized polyethyleneglycol, and any a combination thereof.

4. The composition of claim 1, wherein they hydrogel-forming polymer comprises dextran vinyl sulfone.

5. The composition of claim 1, wherein the hydrogel-forming polymer comprises tetra-thiolated polyethylene glycol.

6. The composition of claim 1, wherein the hydrogel-forming polymer comprises polyethyleneglycol diactrylate.

7. The composition of claim 1, wherein the immune-modulating biomolecule comprises a cytokine.

8. The composition of claim 7, wherein the cytokine comprises a chemokine.

9. The composition of claim 7, wherein the chemokine comprises a chemoattractant.

10. The composition of claim 1, wherein the immune-modulating biomolecule comprises MIP3α, MCP-1, MIP1α, MIP1β, SLC, fMLP, IL-8, RANTES, SDF-1, and any combination thereof.

11. The composition of claim 1, wherein the immune cell recruited or retained is an antigen presenting cell.

12. The composition of claim 1, wherein the antigen-related biomolecule comprises an antigen.

13. The composition of claim 1, wherein the antigen-related biomolecule comprises a nucleic acid.

14. The composition of claim 13, wherein the nucleic acid encodes an antigen.

15. The composition of claim 13, wherein the nucleic acid is an antigen.

16. The composition of claim 1, wherein the antigen-related biomolecule comprises a plasmid containing DNA encoding the Hepatitis B Surface Antigen.

17. The composition of claim 1, further comprising a microparticle containing the antigen-related biomolecule.

18. The composition of claim 1, further comprising a third biomolecule.

19. The composition of claim 18, wherein the third biomolecule comprises a chemokine.

20. The composition of claim 18, wherein the third biomolecule comprises siRNA.

21. The composition of claim 18, further comprising a microparticle containing the third biomolecule.

22. The composition of claim 1, further comprising a microparticle containing the plasmid and further containing IL-10 siRNA.

23. The composition of claim 22, wherein the microparticle further comprises polyethyleneimine and poly(lactic-co-glycolic acid).

24. A method of providing an antigen to an antigen presenting cell in an animal comprising:
administering to the animal at an administration site an immune-modulating hydrogel composition comprising:
at least one hydrogel-forming polymer;
at least one immune-modulating biomolecule operable to recruit or retain the antigen presenting cell; and
at least one antigen-related biomolecule;
forming a hydrogel in-situ from the hydrogel-forming polymer;
recruiting at least one antigen presenting cell to the administration site using the immune-modulating biomolecule; and
inducing phagocytosis of the at least one antigen-related biomolecule by the antigen presenting cell.

25. The method according to claim 24, wherein administering the immune-modulating hydrogel composition comprises intramuscular, subcutaneous, or intradermal injection of the immune-modulating hydrogel composition.

26. The method according to claim 24, wherein forming the hydrogel in-situ occurs within sixty seconds after administration.

27. The method according to claim 24, wherein forming comprises crosslinking the at least one hydrogel polymer.

28. The method according to claim 24, wherein recruiting occurs for at least seventy two hours after the end of administering.

29. The method according to claim 24, wherein the antigen presenting cell is a Langerhans cell or a dendritic cell.
